(19) United States
(12) Patent Application Publication
(10) Pub. No.: US 2022/0176087 A1
(43) Pub. Date: Jun. 9, 2022

(54) System And Method For Treatment Via Bodily Drainage Or Injection

(71) Applicant: NXT Biomedical, LLC, Irvine, CA (US)

(72) Inventors: Robert S. Schwartz, Irvine, CA (US); Stanton J. Rowe, Irvine, CA (US); Alexander Siegel, Irvine, CA (US); Joseph Passman, Irvine, CA (US); Robert C. Taft, Irvine, CA (US); Glen Rabito, Irvine, CA (US)

(73) Assignee: NXT Biomedical, LLC, Irvine, CA (US)

(21) Appl. No.: 17/652,468

(22) Filed: Feb. 24, 2022

Related U.S. Application Data
(63) Continuation of application No. 16/541,077, filed on Aug. 14, 2019.
(60) Provisional application No. 62/718,863, filed on Aug. 14, 2018.
(60) Provisional application No. 62/744,577, filed on Oct. 11, 2018.
(60) Provisional application No. 62/747,644, filed on Oct. 18, 2018.
(60) Provisional application No. 62/804,675, filed on Feb. 12, 2019.
(60) Provisional application No. 62/848,468, filed on May 15, 2019.

(51) Int. Cl. A61M 27/00; A61M 25/01; A61M 25/00; A61M 39/02

(52) U.S. Cl. CPC A61M 27/00; A61M 25/013; A61M 25/0074; A61M 25/10; A61M 2210/101; A61M 2210/12; A61M 39/0208

(57) ABSTRACT

Devices and methods of treating fluid retention caused by congestive heart failure or other conditions resulting in edema, lymphoedema, or significant fluid retention (e.g., deep vein thrombosis, cellulitis, venous stasis insufficiency, or damage to the lymphatic network) are described. Specifically, a treatment device is used to create a passage or cannula between the lymphatic system (or other area of the body) and an external drainage device. This device can be only temporarily located in the patient or can be implanted within the patient for longer periods of time. The physician can safely and reliably remove excess fluid from the body via the device and optionally inject other treatment agents.

This application is a divisional of U.S. patent application Ser. No. 16/541,077 filed Aug. 14, 2019 entitled System And Method For Treatment Via Bodily Drainage Or Injection, which claims benefit of and priority to Provisional Patent Application Ser. No. 62/718,863 filed Aug. 14, 2018 entitled System and Method for Treatment Via Thoracic Duct Drainage or Injection, Provisional Patent Application Ser. No. 62/744,577 filed Oct. 11, 2018 entitled System and Method for Treatment Via Thoracic Duct Drainage or Injection, Provisional Patent Application Ser. No. 62/747,644 filed Oct. 18, 2018 entitled System and Method for Treatment Via Thoracic Duct Drainage or Injection, Provisional Patent Application Ser. No. 62/804,675 filed Feb. 12, 2019 entitled Thoracic Duct Lymphatic Drainage, and Provisional Patent Application Ser. No. 62/848,468 filed May 15, 2019 entitled Pleural and Lymphatic Drainage Systems, all of which are hereby incorporated herein by reference in their entireties.

### BACKGROUND OF THE INVENTION

Chronic and acute congestive heart failure (CHF) generally occurs when the heart is incapable of circulating an adequate blood supply to the body. This is typically due to inadequate cardiac output, which has many causes. In CHF decompensation fluids back up in a retrograde direction through the lungs and venous/lymphatic systems throughout the body, causing discomfort and organ dysfunction. Many diseases can impair the pumping efficiency of the heart to cause congestive heart failure, such as coronary artery disease, high blood pressure, and heart valve disorders.

In addition to fatigue, one of the prominent features of congestive heart failure is the retention of fluids within the body. Commonly, gravity causes the retained fluid to accumulate to the lower body, including the abdominal cavity, liver, and other organs, resulting in numerous related complications. Fluid restriction and a decrease in salt intake can be helpful to manage the fluid retention, but diuretic medications are the principal therapeutic option, including furosemide, bumetanide, and hydrochlorothiazide. Additionally, vasodilators and inotropes may also be used for treatment.

While diuretics can be helpful, they are also frequently toxic to the kidneys and if not used carefully can result in acute and/or chronic renal failure. This mandates careful medical management while in a hospital, consuming large amounts of time and resources. Hence, the ability to treat fluid retention from congestive heart failure without the need for toxic doses of diuretics would likely result in better patient outcomes at substantially less cost.

Fluid retention is not limited only to CHF. Conditions such as organ failure, cirrhosis, hepatitis, cancer, and infections can cause fluid buildup near the lungs, referred to as pleural effusion. The space is lined by two thin membranes (the visceral and parietal pleura) that line the surface of the lungs and the inside of the chest wall. Normally, only a few teaspoons of fluid are located in this space so as to help the lungs to move smoothly in a patient's chest cavity, but underlying diseases can increase this amount. Patients with pleural effusion may need frequent draining directly via a guided needle and catheter introduced directly to the pleura. These procedures are expensive, traumatic, and require hospitalization.

In this regard, what is needed is an improved treatment option for fluid buildup in the body, whether that buildup is caused by CHF, cirrhosis, organ failure, cancer, infections, or other underlying diseases.

### SUMMARY OF THE INVENTION

The present invention is generally directed to devices and methods of treating fluid retention caused by congestive heart failure or other conditions resulting in pleural effusion, edema, lymphoedema, or significant fluid retention (e.g., deep vein thrombosis, cellulitis, venous stasis insufficiency, or damage to the lymphatic network). Specifically, a treatment device is used to create a temporary or permanent passage either directly or via a cannula between the lymphatic system (or other area such as the visceral and parietal pleura around the lungs) and an external drainage device which may be either active (suction) or passive (internal hydrodynamic pressure or gravity). This device can be temporarily located in the patient or can be implanted within the patient for longer periods of time. The physician can safely and reliably remove excess fluid from the lymphatic system via the device and, in some embodiments, inject other treatment agents (e.g., electrolytes, chemotherapeutic agents, inotropes, steroids, antibiotics, or other heart failure, infectious, or cancer treatment agents).

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIGS. 1, 2, and 3 illustrate one embodiment of a drainage system according to the present invention.

FIG. 4 illustrates another embodiment of a drainage system according to the present invention.

FIGS. 5, 6, and 7 illustrate another embodiment of a drainage system according to the present invention.

FIGS. 8, 9, 10, 11, and 12 illustrate another embodiment of a partially implantable drainage system according to the present invention.

FIGS. 13 and 14 illustrate another embodiment of an implantable drainage system according to the present invention.

FIG. 15 illustrates another embodiment of an implantable drainage system according to the present invention.

FIG. 16 illustrates another embodiment of an implantable drainage system according to the present invention.

FIG. 17 illustrates another embodiment of an implantable drainage system according to the present invention.

FIG. 18 illustrates an embodiment of a curved guide catheter according to the present invention.

FIG. 19 illustrates another embodiment of an implantable drainage system according to the present invention.

FIG. 20 illustrates another embodiment of an implantable drainage system according to the present invention.

FIG. 21 illustrates another embodiment of an implantable drainage system according to the present invention.

FIG. 22 illustrates another embodiment of an implantable drainage system according to the present invention.

FIGS. 23, 24, 25, and 26 illustrate another embodiment of an implantable drainage system according to the present invention.

FIG. 27 illustrates another embodiment of an implantable drainage system according to the present invention.

FIG. 28 illustrates another embodiment of an implantable drainage system according to the present invention.

FIG. 29 illustrates another embodiment of an implantable drainage system according to the present invention.

### DETAILED DESCRIPTION

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

The lymphatic system is part of the vascular system and an important component of the immune system, comprising a network of lymphatic vessels that carry lymph directionally toward the heart. The human circulatory system typically processes an average of 10 liters of blood per day into the lymphatics via capillary filtration, which removes plasma while leaving the blood cells. Most of the filtered plasma is reabsorbed directly into the blood vessels, while the remaining plasma remains within the body's interstitial fluid. The lymphatic system provides an accessory return route to the blood for this unabsorbed plasma, as well as other biological materials, known as lymph. Some diseases, such as congestive heart failure, can result in lymphedema or an accumulation of lymph/fluid within the lymphatic system, as well as accumulation of fluid in other parts of the body.

The present invention is generally directed to devices and methods of treating fluid retention caused by congestive heart failure or other conditions resulting in pleural effusion, edema, lymphoedema, or significant fluid retention (e.g., deep vein thrombosis, cellulitis, venous stasis insufficiency, or damage to the lymphatic network). Specifically, a treatment device is used to create a temporary or permanent passage either directly or via a cannula between the lymphatic system and an external drainage device which may be either active (suction) or passive (internal hydrodynamic pressure or gravity). This device can be temporarily located in the patient or can be implanted within the patient for longer periods of time. The physician can safely and reliably remove excess fluid from the lymphatic system (or from other locations such as the lungs) via the device and, in some embodiments, inject other treatment agents (e.g., electrolytes, chemotherapeutic agents, inotropes, steroids, antibiotics, or other heart failure, infectious, or cancer treatment agents).

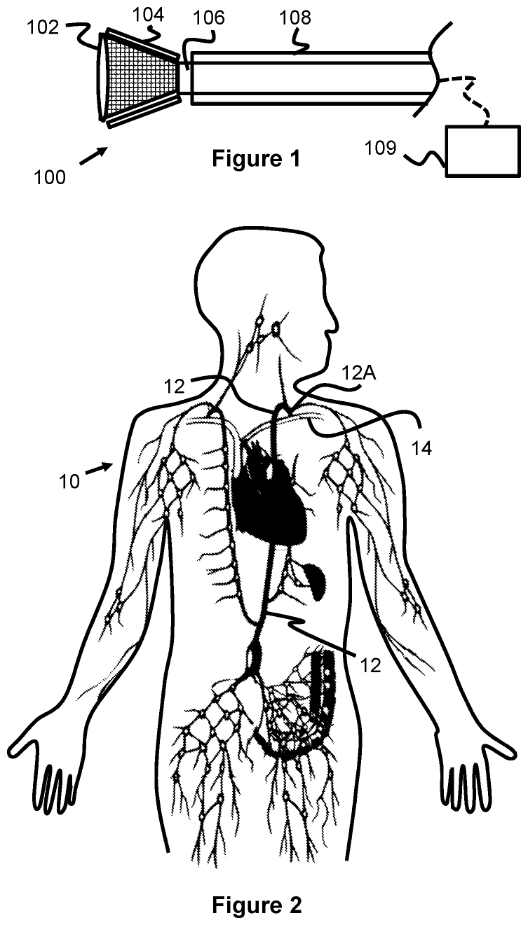

FIG. 1 illustrates one embodiment of a lymphatic treatment device 100 that can be used to apply drainage to remove lymph in a patient's lymphatic system. The device 100 includes a cannula body 106 that is elongated, cylindrical, and has an internal passage therethrough. The distal end of the cannula body 106 has a radially expandable portion 102 while the proximal end of the cannula body 106 is in communication with a drainage device 109 to draw lymph through the cannula body 106.

In one embodiment, the radially expandable portion 102 is composed of braided, shape memory wires (e.g., Nitinol) that are heat set to expand to a conical shape. To enhance efficient flow, a film or fluid-impenetrable layer 104 (e.g., PET or an elastic polymer) is disposed over the braided wires. Both the expandable portion 102 and the cannula body 106 can be composed of a single, tubular braided shape memory layer, such that only the distal portion radially expands when unconstrained (e.g., the cannula body 106 may have one or more polymer layers that restrain its radial expansion). Alternately, only the expandable portion 102 can be composed of braided shape memory wires that are attached to the distal end of the cannula body 106. Alternately, the radially expandable portion 102 can be composed of a laser-cut tube, braided, non-shape-memory wires, an expandable polymer sleeve, or a variety of other structures known in the art. The expandable portion 102 may be cylindrical, conical, or other 3D shapes.

The device 100 may also include a mechanism to control expansion of the expandable portion 102. For example, a longitudinally moveable outer sheath 108 can be initially positioned over the expandable portion 102 to provide restraint. Moving the sheath 108 proximally exposes the expandable portion 102 to allow expansion, while subsequently moving the sheath 108 distally will collapse the portion 102. Alternately, a pull wire may be included within the device 100 to control expansion/contraction of the expandable portion 102, or a balloon expanding technique.

The device 100, as well as any other devices described in this specification, can be connected or positioned at a variety of different locations of the lymphatic system 10, and via numerous different approaches. One particularly desirable treatment location is within the thoracic duct 12, see in FIG. 2. The thoracic duct 12 has one of the largest diameters of all of the lymphatic system and can be accessed relatively easily. For example, the thoracic duct 12 delivers lymph into the left subclavian vein 14 at the thoracic duct valve/ostium 12A. The left subclavian vein 14 can be accessed through the patient's shoulder, leg, or via any other central venous access site via a catheter/guidewire system, allowing a device to then pass through the thoracic duct valve/ostium 12A and into the thoracic duct 12.

The device 100 can be used for treatment via this left subclavian vein approach (or alternately via the femoral vein, internal jugular, right subclavian vein, basilic vein, and brachial vein). For example, a guidewire can be inserted into the left subclavian vein 14 and into the thoracic duct 12. Other devices commonly used for intravascular procedures may also be used. For example, an access sheath can be advanced into the left subclavian vein 14, a guide catheter can be advanced over the first guidewire, the first guidewire can be removed and replaced with a second, smaller-diameter guidewire if necessary, and the device 100 can be delivered over the second guidewire and through the guide catheter.

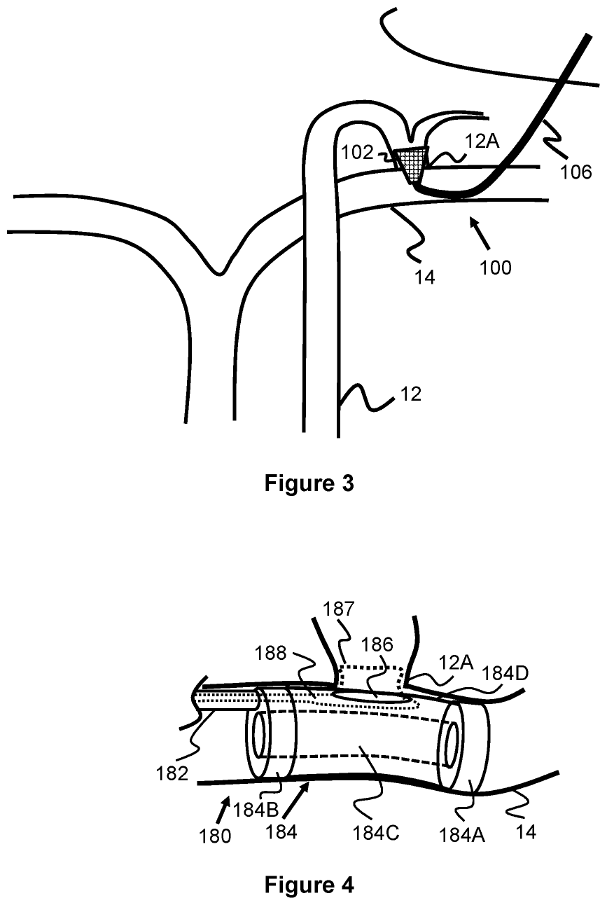

As seen in FIG. 3, once the distal end of the device is positioned at a desired location in the thoracic duct 12 (such as at or just beyond the ostium 12A of the thoracic duct 12), the outer sheath 108 can be moved proximally to expose the expandable portion 102. The expandable portion 102 radially expands to a conical shape and the outer layer 104 allows the expandable portion 102 to form a continuous passage with the thoracic duct 12 externally of the patient. Drainage (e.g. aspiration, suction) via the drainage device 109 is then applied, allowing the lymph to enter the expandable portion 102, the passage of the cannula body 106, and finally outside the patient. When a desired amount of lymph removal has been performed, the outer sheath 108 can be distally advanced (or the cannula body 106 can be proximally withdrawn) to cause radially compression of the expandable portion 102 into the outer sheath 108.

FIG. 4 illustrates an alternate embodiment of a lymphatic treatment device 180 that can be used to apply drainage to remove lymph in a patient's lymphatic system. Unlike the prior device 100 that is positioned into the thoracic duct 12, the device 180 can mostly or completely remain within the left subclavian vein 14 during the lymph removal process.

The device 180 includes a catheter body 182 with a distal expandable portion 184 that expands a drainage opening 186 (which can alternately be used for infusion) against the opening or ostium 12A of the thoracic duct 12, allowing the lymph to drain into a drainage passage 188 that extends through the catheter body 182. In one example, the distal expandable portion 184 comprises a distal circular balloon 184A and a proximal circular balloon 184B that both are inflatable to cause radial expansion of the distal expandable portion 184. Preferably, the balloons 184A, 184B are positioned proximally and distally of the thoracic duct opening and within the left subclavian vein 14, which allows them to expand and isolate the opening of the thoracic duct 12. Once expanded, blood continues to pass through a perfusion passage 184C that opens at each end of the distal expandable portion 184 and this blood perfusion can be maximized by expanding the left subclavian vein 14 to a larger diameter than it would naturally have.

The device 180 also may include a structure that opens the valve leaflets of the thoracic duct 12 at the ostium 12A. In one example, this structure 187 can be an inflatable balloon structure 187 that forms a tubular shape or one or more elongated shapes that project perpendicularly relative to the axis of the device 180. In another example, the structure 187 may be a self-expanding structure composed of memory-shape wires (e.g., a perpendicular braided tubular structure or perpendicular wire loops).

While not shown in the figure, inflation passages preferably extend through the catheter body 182 and distal expandable portion 184 to connect to both balloons 184A, 184B. One advantage of this design is that high fidelity imaging such as fluoroscopy may not necessarily be needed and potentially just Transthoracic Ultrasound (TTE) may be necessary instead. Another advantage is that the balloons may help provide rigid support to the vein and thereby prevent its collapse during the draining process, especially if aspiration is applied.

While the distal expandable portion 184 is illustrated as having a single aperture 186, it may also have a plurality of apertures positioned radially around the distal expandable portion 184 and in between the balloons 184A, 184B. This may obviate the need for a specific rotational orientation.

The distal expandable portion 184 is illustrated as having an outer membrane 184D on which the drainage opening 186 is located. In an alternate example, the outer membrane 184D may not be present and the drainage opening 186 may be located on an inner side of the proximal circular balloon 184B. In other words, the distal expandable portion 184 would be composed of two balloons 184A, 184B, the perfusion passage 184C, and a drainage tube through the proximal balloon 184B. In this regard, the balloons create a closed off space between the perfusion passage 184C and the inner surface of the vein 14. In another example, the distal expandable portion 184 can be composed of a single tubular balloon extending along the entire length of the distal expandable portion 184.

The device 180 may optionally include one or more feelers (e.g., elongated wires extending from a distal end of the device) to provide tactile response for achieving the desired positioning. Depth markers may also be present along the length of the catheter body 182 to further help target a desired position relative to the access point.

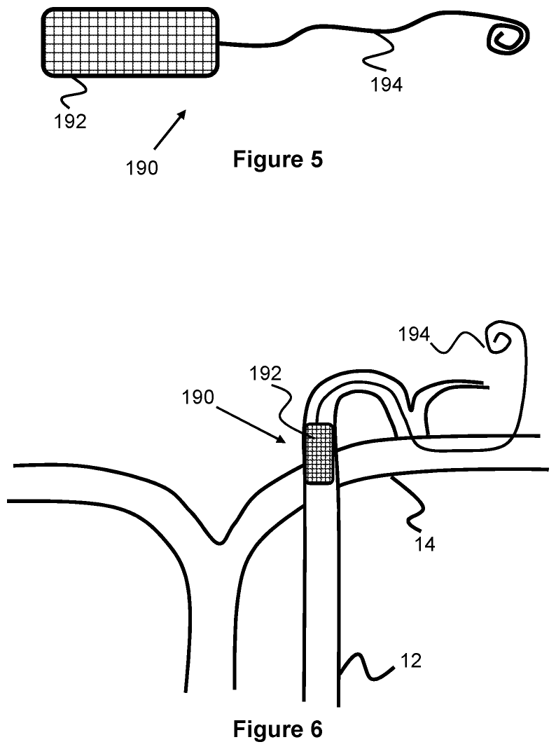

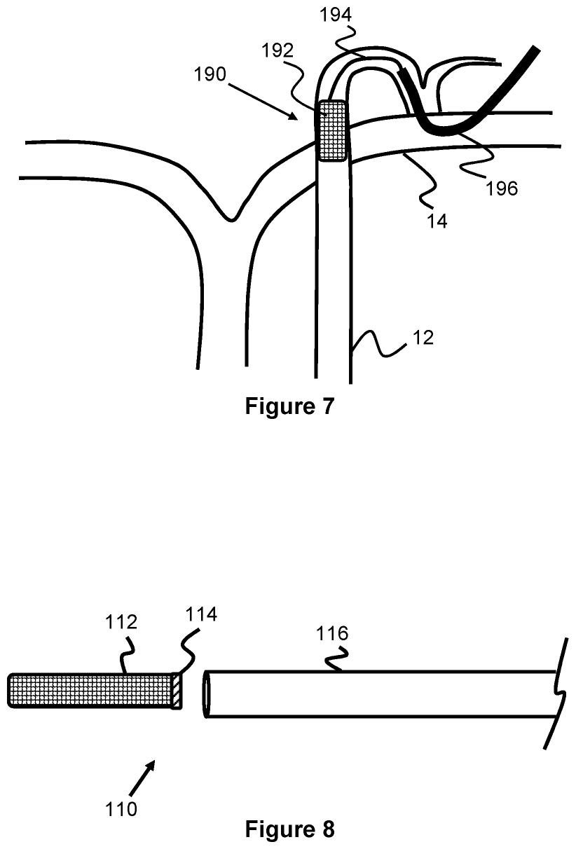

FIGS. 5-7 illustrate an alternate embodiment of a lymphatic treatment device 190 that is only partially implanted to apply drainage to remove lymph in a patient's lymphatic system. The device 190 includes a stent portion 192 and an elongated guidewire 194 that is connected to the stent portion 192 which provides a guide or tracking system for positioning a drainage catheter 196. The stent portion 192 can be a self-expanding or balloon expandable stent-like structure that is expanded within the thoracic duct 12 (e.g., either near the annulus 12A or deeper into the duct 12 as seen in FIG. 6). The guidewire 194 can be tied, looped, welded, glued or otherwise permanently fixed to the stent portion 192 and extends out of the duct 12 and into the left subclavian vein 14. The proximal end of the guidewire 194 may be coiled or may be attached to a larger retrieval structure; both of which may be placed subcutaneously. Since the guidewire 194 may remain within the patient for an extended time, it preferably has a coating that prevents clotting or tissue ingrowth.

When the patient is in need of treatment, the percutaneous location of the proximal end of the guidewire 194 can be accessed and the drainage catheter can be advance over the guidewire 194 and into the thoracic duct 12 to begin drainage. The device 190 can be left in the patient for future treatment sessions. Alternately, the device 190 can be used for a single treatment session and removed from the patient after removal of the drainage catheter 196 and/or the drainage catheter can be permanently connected/implanted in the patient. In one embodiment, the percutaneous access site may include a port or similar device that facilitates multiple accesses of the drainage catheter.

The present invention also contemplates a method of temporarily or permanently holding open the valve leaflets of the thoracic duct 12 near the ostium 12A to allow some chronic drainage into the venous system between drainage sessions. This method includes delivering an implantable device into the thoracic duct 12, positioning the device through the thoracic duct valve so as to maintain the valve in a partially open position and allow fluid from the thoracic duct 12 to move into the left subclavian vein 14.

In one embodiment, the device 190 may allow some chronic drainage into the venous system between drainage sessions with the drainage catheter 196. For example, the stent portion 192 may be positioned at or near the leaflets of the lymph ostium 12A so as to keep the valve to the duct 12 partially open to permit passive drainage into the venous system. Alternately, the drainage catheter 196 may include a plurality of drainage apertures that are located in a proximal portion of the drainage catheter 196 such that they can be positioned in and allow drainage into the venous system. These apertures can be selectively blocked (e.g., by passing another catheter or drainage member directly through the catheter 196 so as to block the apertures. In another embodiment, the guidewire 194 may include an enlargement member that is either fixed to the guidewire 194 or slide over the guidewire 194 and positioned within the valve of the duct 14 to maintain it in a partially open position. It should be understood that other embodiments of this specification can also be used to perform this method of chronic drainage for either a short period of time (e.g., 1-2 hours during a procedure) or chronically via an implanted device (e.g., weeks, months, or years).

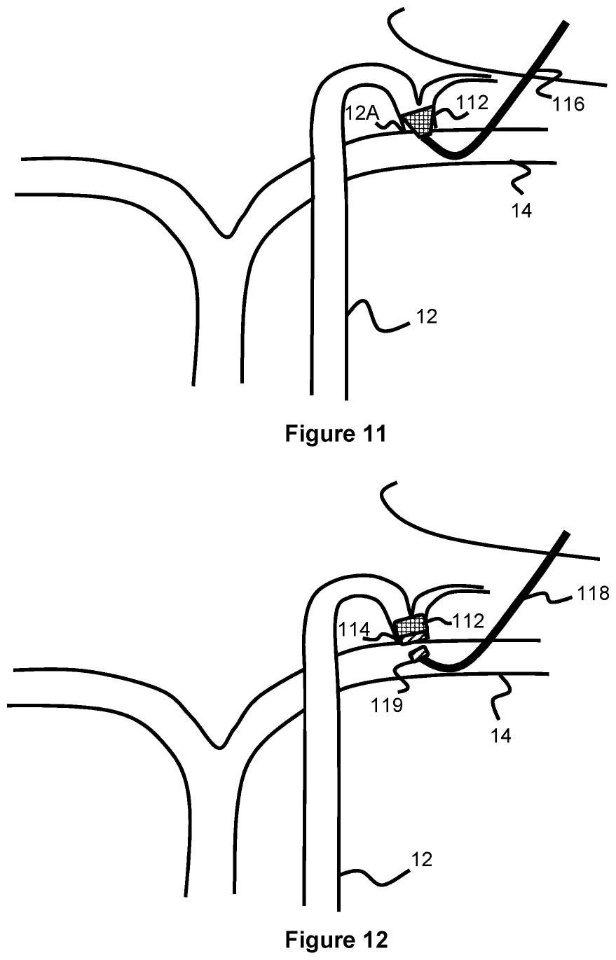

FIGS. 8-12 illustrate another embodiment of a lymphatic treatment device 100 that includes a stent 112 that is implanted into a patient and that can be selectively reconnected to a cannula 118 coupled to a drainage device 109 to remove lymph. As seen in FIG. 11, the stent 112 can be delivered to the thoracic duct 12 via the shoulder and left subclavian vein 14, as well as others described elsewhere in this specification. A stent delivery catheter 116 is first advanced within the thoracic duct 12 until its distal end is located at a desired stent delivery location, such as just beyond the ostium 12A and valve leaflets of the thoracic duct 12. The stent 112 is then exposed and expanded within the thoracic duct 12. This may occur because the stent is radially self-expanding (e.g., composed of braided, heat-set, shape memory wire) and is advanced out of the catheter 116, or is expanded from an integrated or separate inflatable balloon and/or balloon catheter that expands within with stent.

The delivery catheter 116 is then withdrawn from the patient, a cannula 118 is advanced within the thoracic duct 12, and the distal end of the cannula 118 is attached to the proximal end of the stent 112. Alternately, the stent 112 can be placed over the valves of the thoracic duct 12 to maintain it in an open position to achieve chronic drainage in the time between attachment of the delivery catheter, as previously discussed with other embodiments. In this embodiment, the stent 112 and/or threaded portion 114 may include a valve that can be selectively opened by the physician.

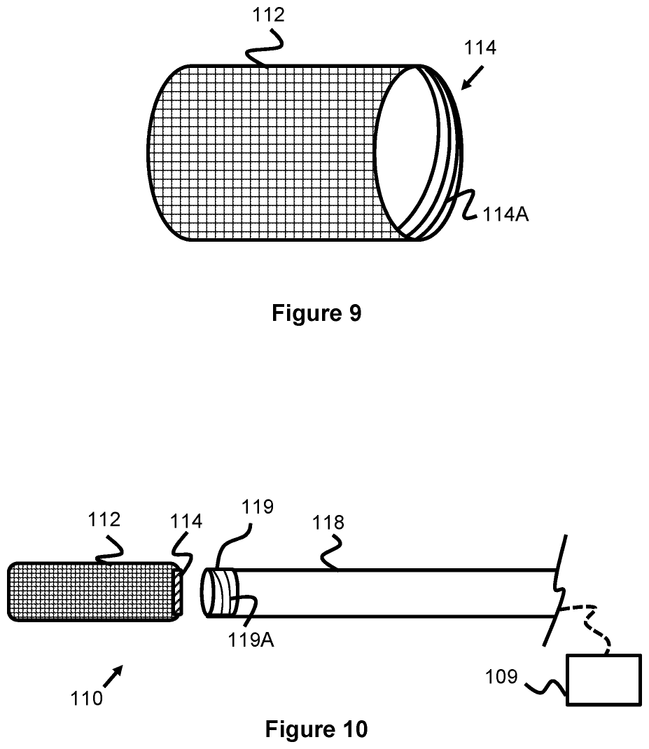

In one embodiment, the stent includes a proximal threaded portion 114. The threaded portion 114 may have threads 114A along its internal diameter, as seen in FIG. 9, or on its outer diameter. The cannula 118 includes a distal threaded portion 119 with a plurality of mating threads on its outer diameter, as seen in FIG. 10, or along its inner diameter and are positioned and configured to engage with threads 114A. As the distal threaded portion 119 contacts the proximal threaded portion 114, the physician rotates the cannula 118, connecting the two together. The threaded portion 114 may be relatively close in diameter to the expanded stent 112 or can have a smaller diameter that causes the stent to form a conical proximal end. In the case of the smaller diameter for the threaded portion 114, it may be desirable to include a fluid-tight outer layer or film (e.g., polymer) to enhance drainage. Once a desired amount of lymph has been removed, the physician can rotate the cannula 118 in the opposite direction to unscrew the stent 112 and the cannula 118 can be removed from the patient. Preferably, radiopaque markers are located at least at/near the threaded portion 114 to provide the physician with guidance as to the location of the stent (though markers at other locations along the stent may also be desirable for subsequent cannulation).

Other attachment mechanisms for the stent 112 are also possible. For example, the proximal end of the stent may include one or more hooks that can latch on to other features of the cannula 118. In another example, the stent 112 may have an annular, flexible ring on its distal end that allows the distal end of the cannula 118 to press against. When the drainage is activated, the drainage force from the cannula 118 will press the distal end of the cannula 118 against the proximal end of the stent 112. Hence, no physical latching/connection mechanism is needed.

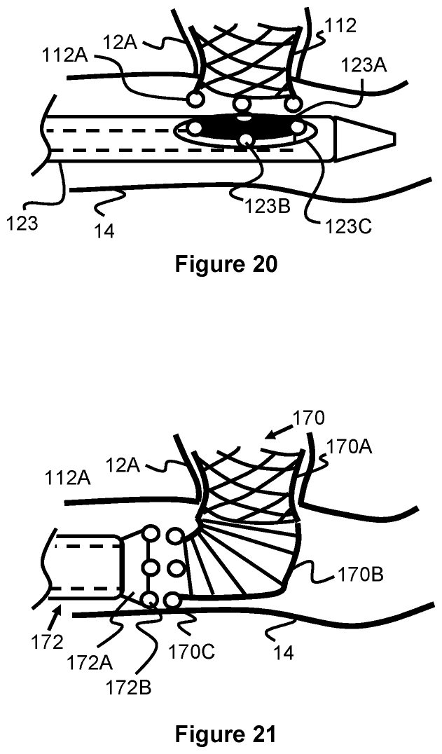

Another example of an attachment system can be seen in FIG. 20 which uses a plurality of magnets to connect the stent and cannula/catheter 123. Specifically, the implanted stent 112 includes a plurality of magnets 112A located at its proximal, exposed end. A catheter 123 has an opening 123A along its side (or alternately on its end) with a plurality of magnets 123B and soft, polymer materials 123C around its circumference. The magnets 112A and 123B attract each other when positioned within proximity of each other and the soft polymer material 123C helps establish a seal with the stent 112 (and optionally with any inner/outer sleeve or layer the stent may have). Optionally, only one set of magnets are needed on either device and the other material can include a ferrous metal that is attracted to the magnets. The magnets 123B can either be located directly on the body of the catheter 123 or can be positioned on the soft polymer materials 123C to allow a small amount of movement. A similar arrangement of polymer material can be located on the end of the stent 112 as well.

FIG. 21 illustrates another example of a magnetic attachment system similar to FIG. 20. However, instead of a stent 112 that is only positioned at the valve/ostium 12A, the stent 170 extends into the left subclavian vein 14. Specifically, the stent 170 includes a first portion 170A with a generally straight profile that expands against the ostium 12A, and a curved second portion 170B that extends from a proximal end of the first portion 170A. The curved second portion 170B can be a generally tubular structure, as seen in the figure, or can be an open curved or concave surface. The proximal end of the second portion 170B includes a plurality of magnets 170C that can attract a plurality of magnets 172B on the distal end of a drainage catheter 172. The plurality of magnets 172B can be mounted on or near a soft, polymer material 172A that helps seal against the proximal end of the second portion 170B (and any inner/outer sleeve or material it may be composed of). In one example, the soft, polymer material 172A forms a conical shape when expanded. Hence, a removable connection to an implanted stent can occur with either a side of a catheter (FIG. 20) or a distal end of a catheter (FIG. 21).

Either of the embodiments of FIGS. 20 and 21 may include a sensor system to determine when the magnets of the stent and the catheter have connected to each other. For example, this can be achieved by allowing the connection of the magnets to complete a circuit path through the catheter, into the stent, and back into the catheter. The catheter may include a power supply on its proximal end that both supplies the power for the circuit and activates an indicator when the circuit is complete.

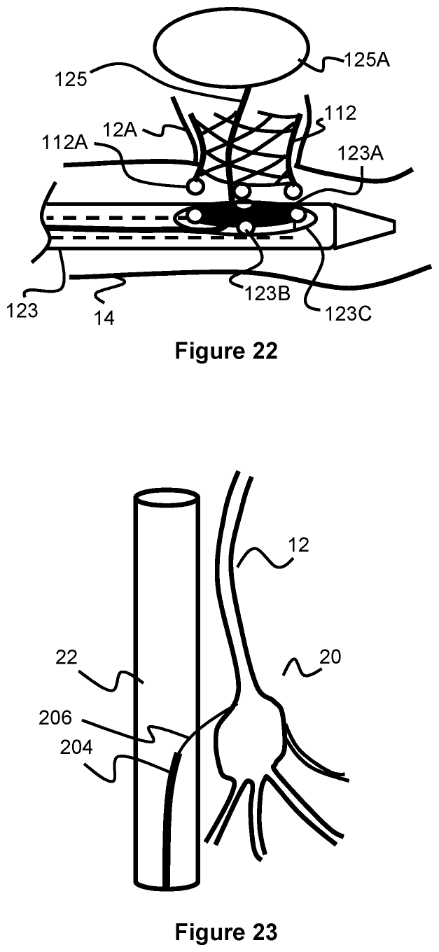

Optionally, a catheter 125 with an inflatable balloon 125A can be inserted through the stent 112 via catheter 125 (or other, similar catheters described herein), as seen in FIG. 22. Rapidly inflating this balloon 125A may help increase the driving pressure within the lymphatic system and thereby increase the drainage rate. Optionally, rapidly deflating this balloon 125A may help decrease the pressure in the proximal most portion of the thoracic duct thereby pull the lymph fluid out of the main thoracic duct. Optionally, if the balloon 125A is navigated deeper, more distal into the main thoracic duct, and then inflated and then pulled back toward the opening of the thoracic duct this would create a vacuum effect thereby drawing out the lymph fluid with the balloon.

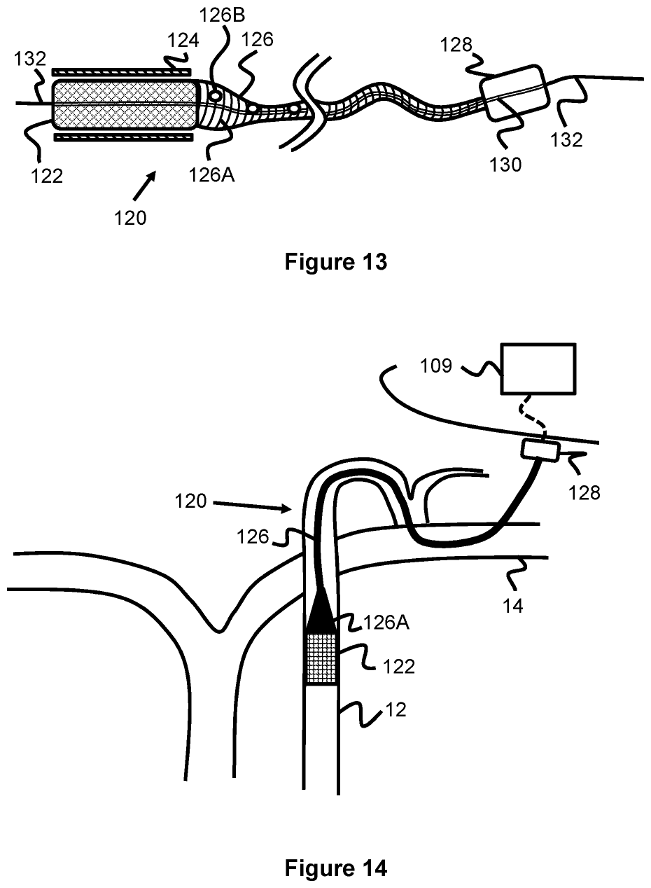

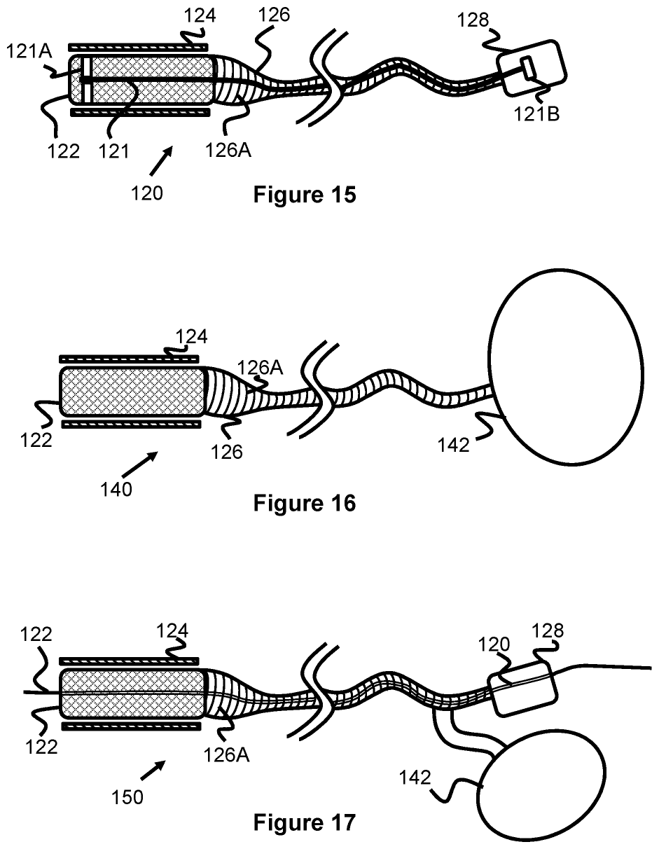

FIG. 13 illustrates one embodiment of an implantable lymphatic device 120 having an elongated tubular portion 126 connected at its distal end to an expandable anchor portion 122 and connected at its proximal end to a port 128. An alternative configuration may be with barbs or hooks on the stent, that can function to stabilize the stent within the thoracic duct by gripping the surrounding tissue and keeping the device firmly attached to the tissue wall. As best seen in FIG. 14, the expandable anchor portion 122 can be expanded and anchored within a portion of the lymphatic system 10, such as in the thoracic duct 12. The elongated tubular portion 126 extends towards the skin, such as near the shoulder, and is sealed by the port 128. In one example, the elongated tubular portion 126 has an expanded diameter that occupies about 40-60% of the diameter of the thoracic duct 12 to allow for normal lymph drainage around the device 120. Often, the thoracic duct 12 can distend to a diameter as large as 15 mm when backed up and under pressure, and therefore a diameter of the elongated tubular portion 126 may be within the range of 5.5 mm to 8.5 mm, or about 7.5 mm to allow for its normal drainage.

In one configuration, the port 128 is located underneath the skin, as seen in FIG. 14. In another configuration, the elongated tubular portion 126 extends out of the skin such that the port 128 is located outside of the body. The external positioning may be particularly useful for relatively quicker, temporary uses of the device, such as only when the patient is admitted to a hospital. The distal end of the elongated tubular portion 126 includes a conical shape that outwardly tapers to the anchoring portion 122. Alternately, the anchoring portion 122 can have a proximal end that tapers proximally to the diameter of the elongated tubular portion 126.

The expandable anchor portion 122 can have a cylindrical shape that can radially expand from a smaller compressed diameter to a larger expanded diameter. The anchor portion 122 can be formed from a plurality of woven/braided metal wires or from a laser-cut cylinder. The anchor portion 122 can be composed of a shape memory material, such as Nitinol, that self-expands to its radially expanded diameter when unconstrained. Alternately or in addition to the self-expansion, a balloon catheter can be used to expand the anchor portion 122 when positioned within the thoracic duct 12. In one example, the anchor portion 12 expands to a diameter within a range of 3 mm to 8 mm.

The anchor portion 122 can optionally include a cylindrical cover 104 that is disposed over the outer surface of the anchor portion 122. This cover 104 may reduce friction between the anchor portion 122 and the delivery device (e.g., a delivery catheter) and further covers any apertures present in the anchoring portion 122 (e.g., caused by braided wires) to enhance drainage pressure. In one example, the cover 124 is composed of a biocompatible polymer film such as PET or an elastic polymer.

The elongated tubular portion 126 is preferably structured to be both flexible and kink resistant. In one embodiment, the tubular portion 126 is composed of a helical wire coil 126A (either monofilar or multifilar) that is attached, embedded, or sandwiched between biocompatible polymer layers that prevent leakage of fluid. For example, a wire can be tightly woven around a cylindrical mandrel and heat set, and then one or more fluid impenetrable layers can be attached to the coil. Use of the helical coil 126A provides additional wall strength that may better resist collapsing when suction is applied, vs. non-wire reinforced tubing. In another embodiment, a tubular braided wire structure can be used instead of or in addition to the wire coil 126A. Optionally, a plurality of drainage holes 126B can be spaced at various intervals along the length of the tubular portion 126, extending with the interior drainage passage and thereby allowing the tubular portion 126 to intake fluid, either in addition to the opening at a distal end of the tubular portion 126 or instead of the distal opening. In one example, multiple apertures can be included at locations around the circumference of the tubular portion and can be spaced apart longitudinally from each other at increments of 0.1 cm to 3 cm. In one example, the tubular portion 126 has a length between 2 cm and 64 cm, and has apertures 126B at intervals along its entire length.

The distal end of the tubular portion 126 is connected to a proximal end of the anchor portion 122 and is at least partially positioned within the thoracic duct 12 so as to create a continuous passage between the duct 12 and the port 128 at its proximal end. In one example, the tubular portion 106 has a length within the range of 0.5 m to 1 m.

The port 128 may be composed of a rigid tubular or circular structure with a self-sealing middle or inner portion that allows for penetration by a syringe needle. For example, the self-sealing portion may be composed of a flexible silicone or similar polymer. As previously discussed, the port 128 can have a relatively thin shape to allow for implantation under the skin of the patient or can have a relatively narrow shape if positioned external to the skin. In an example use where the port 128 is located outside the body or is intended to be directly accessed by cutting the patient's skin for treatment, the port 128 may include a valve that can be opened/closed by the physician (e.g., a Tuohy-Borst style valve).

As seen in FIG. 13, the device 120 may also include a guidewire passage 130 along its length for allowing a guidewire 132 to pass through. This passage 120 may assist in delivering the device 120 to the thoracic duct 12. It may be desirable to leave the guidewire 132 within the patient after implantation of the device 132 to help prevent the passage 130 from clogging with protein and other material.

In an example use where the port 128 is located outside the body or is intended to be directly accessed by cutting the patient's skin for treatment, the device can include a removable stylet 121 that blocks the passage of the device 100 when not in use, but can be removed during a treatment procedure. The stylet 121 prevents proteins and other material from accumulating in and clogging up the passage of the device 120. Preferably, the stylet 121 has an elongated flexible body that conforms to the position/configuration of the implanted device 120. The distal end of the stylet 121 includes an annular seal 121A that is preferably composed of a resilient, compressible material that expands against the inner surface of the device 120. For example, a sponge material, silicone, or even a hydrogel material can be used for the seal 121A. The stylet 121 can be of a length so as to position the seal 121A in either the anchor portion 122, the distal conical portion of the elongated tubular portion 126, or in the more uniform portion of the elongated tubular portion 126.

In a separate configuration, a central cannula can be advanced from proximal to distal down the fluid lumen and left in place to block flow and limit subsequent obstruction if or when the device is left in place for longer time periods. The cannula/stylet can be made with a soft distal end which is capable of compression as it is in the lumen so that fluid is actively excluded. In another embodiment, the blocking stylet can be advanced out of the distal catheter, which permits expansion, and when pulled retrograde toward the distal tip blocks fluid. This configuration can be used if the device is left implanted for long time periods where maintaining patency is of substantial concern.

As with any of the embodiments of this specification, the device 120 can be delivered by accessing the left subclavian vein 14 through the shoulder or any other route to the central venous system and then advancing to the thoracic duct 12. The delivery procedure can include initially advancing a first guidewire to a desired thoracic duct location, inserting a sheath into the left subclavian vein 14, advancing a guide catheter over the first guidewire, replacing the guidewire with a smaller, second guidewire, and delivering the device 120 via a delivery catheter (such as delivery catheter 116) through the guide catheter. If the port 128 is to remain under the patient's skin, a space can be hollowed/created within the patient's shoulder.

FIG. 16 illustrates an alternate embodiment of an implantable lymphatic treatment device 140 that is generally similar to the previously described device 120, including the delivery technique. However, instead of a port, the proximal end of the elongated portion 126 is connected to a reservoir 142 in which lymph accumulates. The reservoir 142 can be composed of a fluid impenetrable material that is completely enclosed and self-seals after being penetrated with a needle (e.g., for drainage or delivery of a treatment drug). For example, the reservoir 142 can be composed of flexible polymer such a silicone rubber, polyethylene, polyurethane, Polyether ether ketone (PEEK), or the like. It may also be made by a 3-dimensional metal filament or fiber weave that is coated to make it fluid-proof. The reservoir 142 is also preferably implanted near the skin so that a physician can easily access it with a needle through the skin when necessary for treatment.

FIG. 17 illustrates another similar variation of an implantable lymphatic treatment device 150 that includes both a port 128 and a reservoir 142 in communication with the elongated portion 126. In this respect, the physician can use a needle to remove/add via the reservoir 142 for treatment or can access the port 128 for treatment (e.g., especially if the port 128 is external to the patient, allowing for greater thoracic duct access).

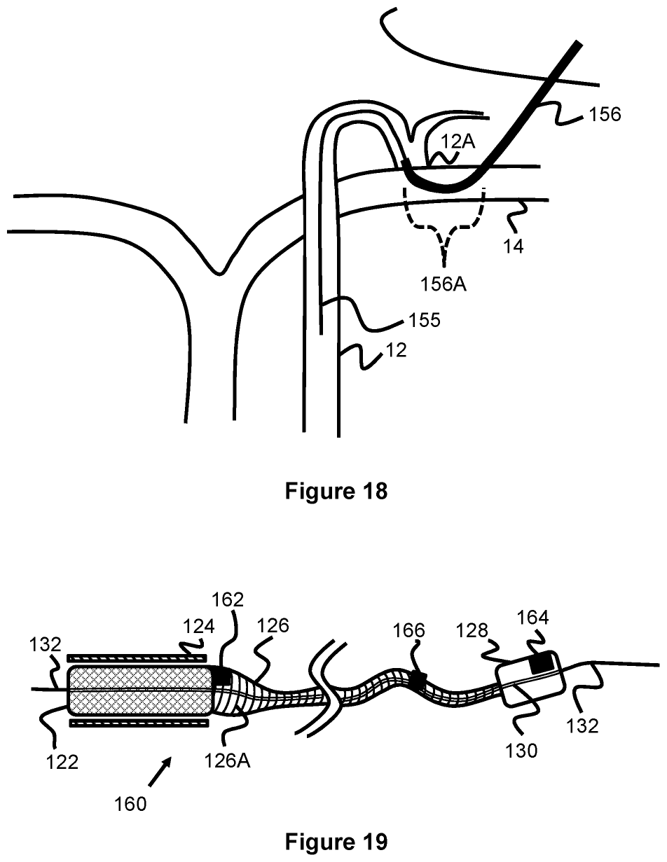

As previously discussed, it may be desirable during a procedure to advance a guide catheter over a guidewire placed in the left subclavian vein 14 and thoracic duct 12. FIG. 18 illustrate one such guide catheter 156 placed over a guidewire 155 that has a distal portion 156B that is biased to a curved shape. This curved shape helps the guide catheter 156 move and transition from entering the left subclavian vein 14 and into the valve/ostium 12A of the thoracic duct 12. In one example, the distal portion 156B has a curve within a range of about 90 degrees over a length within a range of about 1-3 cm.

Any of the embodiments of this specification may also include sensors for monitoring various aspects of a patient, such as pressure sensors, flow sensors, cellular material sensors, protein content sensors, and gene analysis sensors. For example, FIG. 19 illustrates an implantable lymphatic device 160 that is similar to the previously described embodiments. However, it includes a distal sensor 162. The sensor 162 can be located in the anchor portion 122, at the distal end of the elongated portion 126, at the port 128, or at any other location along and within the device.

While the sensor 162 can measure the environment within the thoracic duct 12, a second sensor 166 can also be positioned at a distance along the outside of the device 160 to measure data within the left subclavian vein 14 (or whatever vessel the device is positioned within to reach the thoracic duct 12). Again, pressure sensors, flow sensors, cellular material sensors, protein content sensors, and gene analysis sensors can be used here. In this respect, the device 160 can measure, for example, both thoracic duct pressure and blood pressure.

The sensors 162 and 166 are connected, e.g. via embedded wires, to a communication device 164 in the port 128. The communication device 164 may include a microcontroller (or similar processor), memory for data storage, and a wireless communication transceiver (e.g., Bluetooth, wifi), which allows it to receive and at least temporarily store sensor data, and then transmit that data to an external device.

The device 160 allows for numerous different methods of use. For example, if sensor 162 is a pressure sensor, a physician may draw off lymphatic fluid while monitoring the pressure. Once the lymphatic pressure reaches a desired level, the fluid withdrawal procedure may be stopped.

In another example, a patient could monitor their pressure at home by connecting the device 160 to their phone or similar device. An app on the device/phone can then be used to alert the patient that their lymphatic pressure has reached a level requiring withdrawal and/or can be sent to a nursing station or cloud site for a physician or nurse to determine if further treatment is necessary. The patient can then be contacted by the medical facility monitoring the pressure to schedule an appointment for fluid withdrawal.

While many patients may benefit from lymph drainage as previously described, this type of drainage is challenged by the loss of proteins and lymphatic cells which may result in compromised immune function. One approach to reducing this protein loss while still providing drainage is to create a shunt from the patient's lymphatic system to a low-pressure zone of their body. For example, the shunt may connect to the bladder, the small bowel, the right atrium, or the right ventricle.

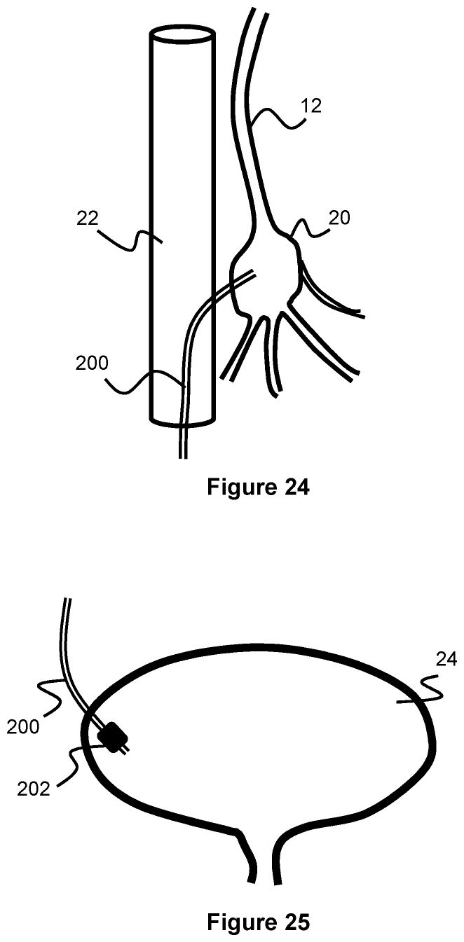

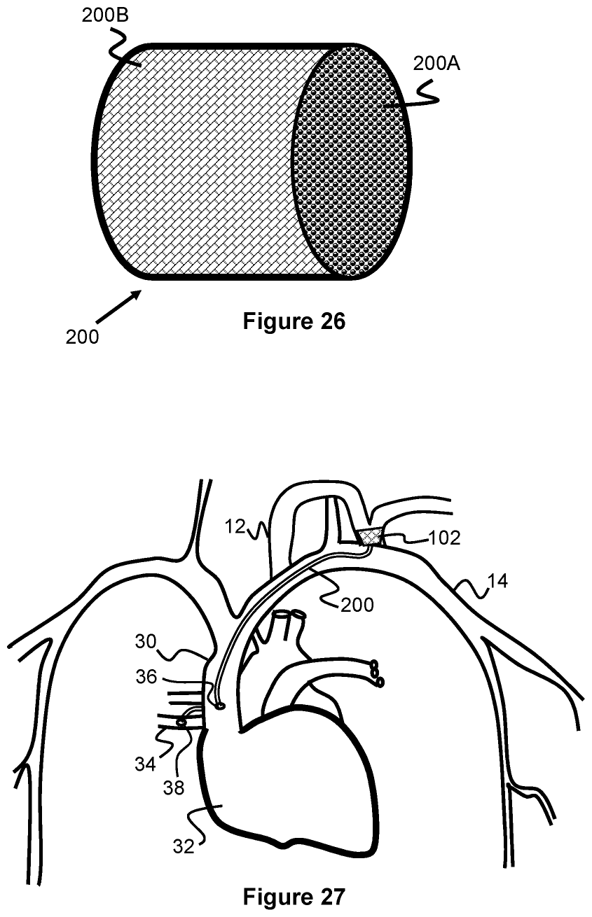

FIGS. 23-26 illustrate various aspects of one example shunt 200 and its use within a patient. Turning first to FIG. 23, the patient's inferior vena cava 22 is accessed via the femoral vein, allowing a catheter 204 to be advanced to a location near the cisternae chyli 20 of the lymph system. Next, a needle 206 is advanced out of the catheter 204 in a direction to puncture both the inferior vena cave and the cisternae chyli 20. Once the needle 206 is in place, a shunt or dialysis catheter 200 is advanced over the needle 206 so that its distal end is located in the cisternae chyli. The distal end of the catheter may have a geometry or an anchor that allows fixation of the distal end of the catheter within the cisternae chyli or another portion of the lymphatic system. For example, the distal end may include an inflatable balloon anchor or a stent-like, self-expanding anchor. Next, the distal end of the shunt 200 is implanted in a drainage location in the body. This location can be the bladder, as seen in FIG. 25, the duodenum, intestine, a subcutaneous port or artificial subcutaneous reservoir, or similar location. Similarly, this end of the catheter may require a geometry or anchor that allows for fixation and hemostasis of the catheter inside the drainage location within the body. For example, the distal end may include an inflatable balloon anchor or a stent-like, self-expanding anchor. In the case of use in the bladder 24 or duodenum, a one-way valve 202 can be included near the proximal end of the shunt 200 to only allow fluid to into that location, but not back up to the lymph system.

In one embodiment seen in FIG. 26, the shunt 200 comprises a plurality of dialysis fibers 200A which are generally known in the art. In one example, these fibers are hollow and have a diameter of about 200 micrometer, which allow the walls of the hollow fibers to function as the dialysis membrane. The fibers can be composed of various materials, such as cellulose-based materials and synthetic polymers.

The outer tubular wall 200B of the shunt 200 is preferably comprised of a water/fluid proof material (e.g., polyurethane) that prevents non-lymph fluids from being absorbed. The wall 200B may also be composed of a porous structure (e.g., 75-100 micrometer diameter) that may help create arterial endothelial and new intimal growth with the surrounding tissue. The shunt 200 may be implanted temporarily, for a short-term, or for a long term. In this example, the outer tubular wall 200B is configured as a chronic implant into interface with friable native tissues and tubes. In this example, the tubular wall 200B is composed of a porous cylindrical structure that has strong radial components preventing its collapse. It is further highly compliant and may be any spring structure or a cross-weave configuration that allows for bending and prevents collapse. the 75-100 micrometer diameter helps permit a pannus formation around the wall 200B, developing an endothelium and thus creating a completely biological surface.

FIG. 27 illustrates another example use of a shunt 200 within a patient to drain a patient's lymph system into the right pulmonary vein 34. One end of the shunt 200 may include an expandable stent portion 102 and can be fixed within the thoracic duct 12 as previously described in this specification. The shunt 200 is positioned through the left subclavian vein 12 and into the superior vena cava 30, just above the heart 32. The shunt 200 then is positioned through the wall of the superior vena cava 30 and into the right pulmonary vein 34. In patients with poor lymph drainage, the pressure in the right pulmonary vein 34 may be relatively lower than that of the thoracic duct 12 and therefore may provide better lymph system drainage. Optionally, a one-way valve may also be included in the shunt to help maintain fluid flow into the right pulmonary vein 34. Features may be added to the ends of the shunt to aid in hemostasis and anchoring. These features may include a self-expanding or balloon-expanding stent like structures made from metal or polymers.

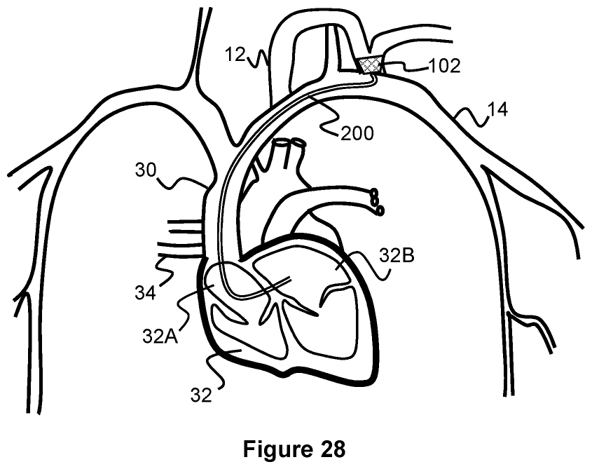

FIG. 28 illustrates another example use of a shunt 200 that is similar to that of FIG. 27 but instead drains into the left atrium 32B. Again, the shunt 200 is anchored in the thoracic duct 12 via a stent portion 102 and is positioned through the left subclavian vein 14 and into the superior vena cava 30. From there, the shunt 200 enters the right atrium 32A, is positioned through the septum, and terminates in the left atrium 32B. Hence, the thoracic duct 12 can drain into the relatively lower pressure region of the left atrium 32B. Features may be added to the ends of the shunt to aid in anchoring. These features may include a self-expanding or balloon-expanding stent like structures made from metal or polymers.

While the embodiments of this specification have been described mostly for drainage of the lymph system, it should be understood that these embodiments and methods can be used for drainage of other conditions. One example is a pleural effusion, which is when an unusually large amount of fluid builds up around the lungs and within the pleural spaces due to a number of different underlying medical conditions. This space is lined by two thin membranes (the visceral and parietal pleura) that line the surface of the lungs and the inside of the chest wall. Normally, only a few teaspoons of fluid are located in this space so as to help the lungs to move smoothly in a patient's chest cavity, but underlying diseases can increase this amount. Pleural effusion is frequently caused by organ failure, cancer, and infections. Patients with pleural effusion may need frequent draining directly via a guided needle and catheter introduced directly to the pleura. These procedures are expensive, traumatic, and require hospitalization.

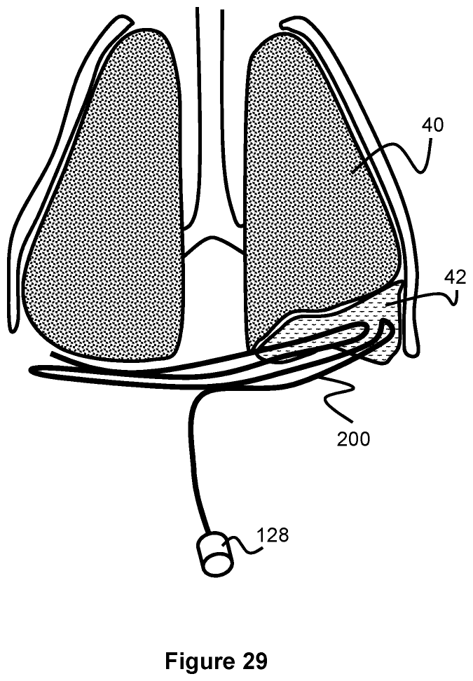

FIG. 29 illustrates another treatment approach for pleural effusion in which a shunt 200 or alternately an implanted drainage catheter is used to drain the pleura to a subcutaneous port 128 or to another location in the body, such as the bladder or small intestine. In one embodiment, a delivery catheter is advanced through the femoral vein and into the vena cava. A needle of the catheter or located in the catheter is advanced just above the junction of the inferior vena cava and towards the diaphragm into the pleural space.

Once the delivery catheter is located within the pleural space, the shunt 200 or drainage catheter can be advanced into the pleural space 42 (and especial into the areas retaining excess fluid). Depending on how and where the fluid is being retained, the shunt 200 may be positioned back and forth along the floor of the diaphragm beneath the lungs 40 (e.g., in loop formations) or along just a portion of the pleural space.

The structure of the shunt 200 may vary depending on where the shunt 200 drains to. For example, if the shunt 200 drains to a subcutaneous port 128, it may have a generally hollow, tubular passage with a plurality of drainage apertures located along the portion positioned below the lungs 40. In another example, if the shunt 200 drains to the intestine, bladder, or other internal location, the shunt 200 may be composed of dialysis fiber, as discussed in the embodiment of FIG. 27 and may further include a one-way valve to restrict the directly of fluid movement. Either of these shunt 200 embodiments can allow the excess fluid to drain but also may allow reabsorption of important biological substances in the fluid that would otherwise be lost.

In any of the previous embodiments, the anchoring portion 122 or stent 112 can include anti-thrombus and/or anti-cellular coatings. These may help reduce obstruction of the device or cellular overgrowth.

While the embodiments of this specification have primarily been described in terms of removing lymph from the lymphatic system, it should be understood that treatment agents can also be added to the lymphatic system via any of the described devices. Once a device has been inserted and/or implanted, a treatment agent can be injected into the device accordingly (e.g., into the cannula, port, or lumen). For example, treatment agents may include electrolytes, chemotherapeutic agents, steroids, antibiotics, or other heart failure or cancer treatment agents.

In any of the embodiments that include an implantable device, it should be understood that they can be removed at a later date. For example, a recovery sheath can be advanced over the implant, causing it to compress. The sheath and device can then be removed from the patient.

While the embodiments of this specification have been described as being implanted via the shoulder and left subclavian vein, other access points are also possible. For example, the device can be advanced via the groin to the subclavian vein and thoracic duct.

In another aspect of the present invention, any of the devices of this specification can be used to withdraw lymphatic fluid to screen for malignant cells or other cells indicating internal disease states, such as metastatic cancers.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A method of removing excess fluid from a lymphatic system of a patient, comprising:
radially expanding a tubular anchor in a thoracic duct; the tubular anchor being attached to and extending distally beyond a distal end of an elongated tubular portion and comprising a plurality of woven wires or a laser-cut cylinder;
deploying the elongated tubular portion;
positioning a proximal end of the implantable drainage device adjacent to the patient's outer skin surface so as to allow the proximal end to be subcutaneously accessible or positioned outside of the patient.

2. The method of claim 1, wherein the distal end of the elongated tubular portion has a conical shape that increases in diameter towards the anchor.

3. The method of claim 1, wherein the proximal end is a subcutaneously accessible port.

4. The method of claim 3, wherein the subcutaneously accessible port further comprises a valve configured for accessing a drainage passage of the elongated tubular portion.

5. The method of claim 1, wherein deploying the elongated tubular portion further comprises occupying 40-60% of the diameter of the thoracic duct with the elongated tubular portion.

6. The method of claim 1, wherein deploying the elongated tubular portion further comprises occupying about 5.5 mm to 8.5 mm of the diameter of the thoracic duct with the elongated tubular portion.

7. The method of claim 1, wherein the elongated tubular portion further comprises a coiled helical wire positioned between biocompatible polymer layers.

8. The method of claim 1, further comprising placing an elongated flexible body within a drainage passage of the elongated tubular portion so as to occlude the drainage passage.

9. The method of claim 8, further comprising a seal disposed on an end of the elongated flexible body; the seal being configured to contact against the inner surface of the drainage passage of the elongated flexible body.

10. The method of claim 1, wherein the proximal end of the implantable drainage device is a subcutaneously accessible reservoir.

11. The method of claim 1, wherein the elongated tubular portion comprises a plurality of apertures connecting to a drainage passage extending through the elongated tubular portion.

12. The method of claim 1, further comprising a polymer film cover disposed over an outer surface of the anchor.
